(19) United States
(12) Patent Application Publication
(10) Pub. No.: US 2010/0087476 A1
(43) Pub. Date: Apr. 8, 2010

(54) PHARMACEUTICAL COMPOUNDS

(75) Inventors: Andrew John Duffield, Berkhamsted (GB); Neil Andrew Williams, Cheddar (GB)

Correspondence Address: SCHWEGMAN, LUNDBERG & WOESSNER, P.A., P.O. BOX 2938, MINNEAPOLIS, MN, 55402, US

(73) Assignee: Biovail Laboratories International (Barbados) S.R.L., Christ Church (BB)

(21) Appl. No.: 12/555,732

(22) Filed: Sep. 8, 2009

(30) Foreign Application Priority Data
Sep. 8, 2008 (GB) 0816370.1

(51) Int. Cl. A61K 31/4375; A61P 11/06

(52) U.S. Cl. 514/294

(57) ABSTRACT

This application describes methods of treatment and/or prophylaxis of asthma that involve administration of dihydrotetrabenazine, and isomers and/or pharmaceutically acceptable salts thereof.

### RELATED APPLICATIONS

This application claims benefit of the filing date of UK Patent Application No 0816370.1 filed Sep. 8, 2008, the contents of which are specifically incorporated herein in their entirety.

### FIELD OF THE INVENTION

This invention relates to the use of dihydrotetrabenazine in the prophylaxis or treatment of asthma.

### BACKGROUND OF THE INVENTION

Asthma is one of the most common chronic medical conditions in the developed world and is responsible for many thousands of deaths each year. Asthma can be characterized as an obstruction of the airways which leads to chest tightness, wheezing, coughing and difficulties in breathing. Typical triggers for asthma include allergens, strenuous exercise, cold air, infections, exposure to atmospheric irritants and strong odors. The pathogenesis of asthma is varied and there are several biological pathways involved in the development of asthma (see R. Balkissoon, Prim. Care Clin. Office Pract., 35 (2008) 41-60).

Asthma can be classified according to clinical phenotype as follows:
Allergic vs. non-allergic asthma
Late- vs. early-onset asthma
Exercise-induced asthma
Nocturnal asthma
Cough variant asthma
Work-related asthma
   Work aggravated asthma
   Occupational asthma
      Large molecular weight (classic IgE)
      Low molecular weight (non-IgE)
      Reactive airways dysfunction syndrome
Inner city (urban) asthma
Infection-related asthma

### SUMMARY OF THE INVENTION

This invention relates to the use of cis-dihydrotetrabenazine in the prophylaxis and treatment of asthma.

Accordingly, one aspect of the invention is a method for the prophylaxis or treatment of asthma in a patient, which method comprises administering to the patient a therapeutically effective amount of a 3,11b cis-dihydrotetrabenazine, or an isomer and/or pharmaceutically acceptable salt thereof, to thereby treat asthma.

The asthma can be any one or more of asthma types selected from the group consisting of allergic asthma, non-allergic asthma, late onset asthma, early-onset asthma, exercise-induced asthma, nocturnal asthma, cough variant asthma, work-related asthma, work aggravated asthma, occupational asthma, asthma induced by large molecular weight compounds, IgE-dependent asthma, asthma induced by low molecular weight compounds, IgE-independent asthma, reactive airways dysfunction syndrome, and inner city or urban asthma. In some embodiments, the asthma is allergic asthma or exercise-induced asthma. In other embodiments, the asthma is infection-induced asthma.

The 3,11b-cis-dihydrotetrabenazine, or a pharmaceutically acceptable salt thereof, can a 2S,3S,11bR isomer of 3,11b-cis-dihydrotetrabenazine having the formula (Ia):

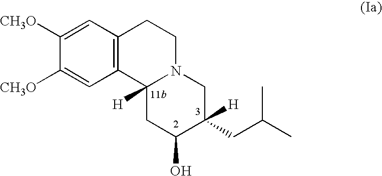

or a pharmaceutically acceptable salt thereof.

In other embodiments, the 3,11b-cis-dihydrotetrabenazine, or a pharmaceutically acceptable salt thereof, can be a 2R,3R,11bS isomer of 3,11b-cis-dihydrotetrabenazine having the formula (Ib):

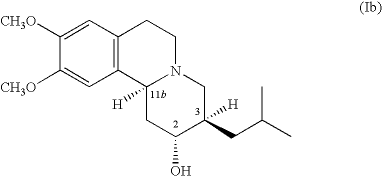

or a pharmaceutically acceptable salt thereof.

In further embodiments, the 3,11b-cis-dihydrotetrabenazine, or a pharmaceutically acceptable salt thereof, can be a 2R,3S,11bR isomer of 3,11b-cis-dihydrotetrabenazine having the formula (Ic):

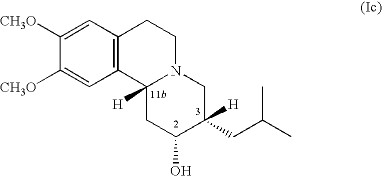

or a pharmaceutically acceptable salt thereof.

In other embodiments, the 3,11b-cis-dihydrotetrabenazine, or a pharmaceutically acceptable salt thereof, can be a 2S,3R,11bS isomer of 3,11b-cis-dihydrotetrabenazine having the formula (Id):

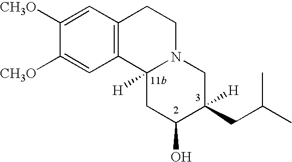

or a pharmaceutically acceptable salt thereof.

Combinations of 3,11b-cis-dihydrotetrabenazine isomers can also be employed in the methods and compositions of the invention.

The 3,11b-cis-dihydrotetrabenazine (or isomer thereof) can be a free base or any pharmaceutically acceptable salt. For example, the pharmaceutically acceptable salt of 3,11b-cis-dihydrotetrabenazine (or isomer thereof) can be an acid addition salt. One example, of such an acid addition salt is a methane sulphonate salt of 3,11b-cis-dihydrotetrabenazine.

Another aspect of the invention is 3,11b-cis-dihydrotetrabenazine, or a pharmaceutically acceptable salt thereof, for use in the prophylaxis or treatment of asthma.

Another aspect of the invention is the use of 3,11b-cis-dihydrotetrabenazine, or a pharmaceutically acceptable salt thereof, for the manufacture of a medicament for the prophylaxis or treatment of asthma.

Another aspect of the invention is a method for the prophylaxis or treatment of asthma in a patient, which method comprises administering to the patient a therapeutically effective amount of a 3,11b cis-dihydrotetrabenazine, or a pharmaceutically acceptable salt thereof.

The types of asthma for which the 3,11b cis-dihydrotetrabenazines of the invention can be used include any one or more types selected from:
Allergic asthma
Non-allergic asthma
Late onset asthma
Early-onset asthma
Exercise-induced asthma
Nocturnal asthma
Cough variant asthma
Work-related asthma
   Work aggravated asthma
   Occupational asthma
      Large molecular weight (classic IgE)
      Low molecular weight (non-IgE)
      Reactive airways dysfunction syndrome
Inner city (urban) asthma
Infection-related asthma

### BRIEF DESCRIPTION OF THE DRAWINGS

FIG. 1 illustrates the effect on differential cell counts in bronchial lavage fluid in a murine model of asthma following different treatments.

FIG. 2 illustrates the effect of different treatments on respiratory distress (Pen H) values) in a murine asthma model following exposure to increasing concentrations of methacholine.

FIG. 3 illustrates the histopathology scores in lungs from OVA-sensitised mice following different treatment regimes.

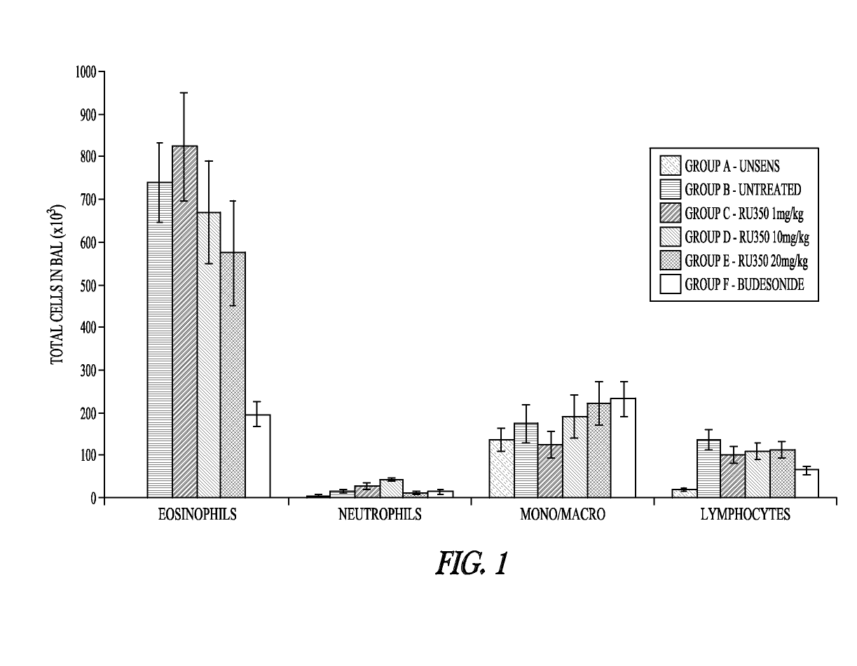

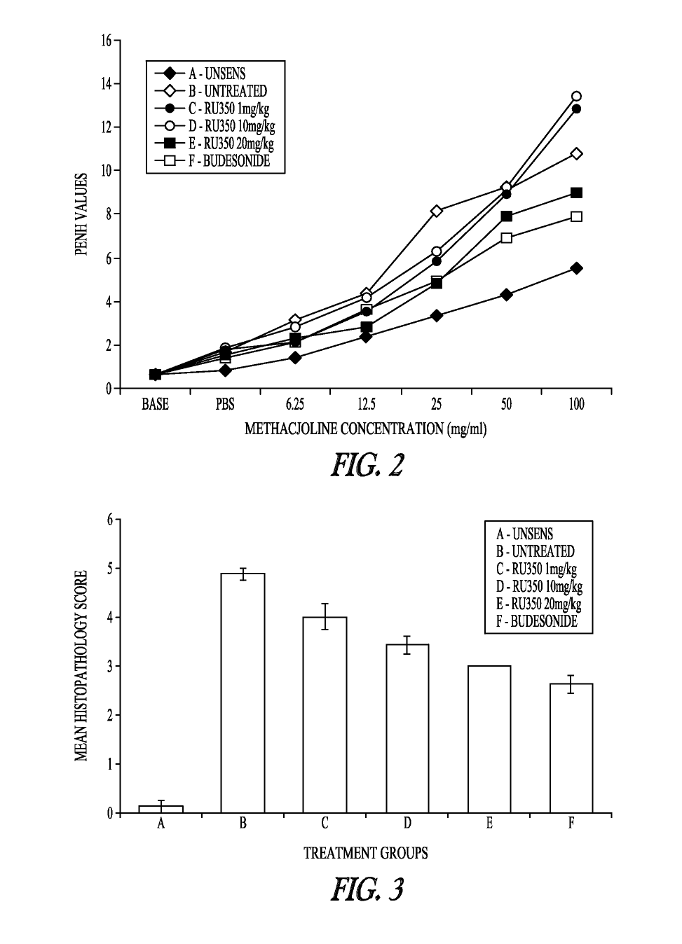

### DETAILED DESCRIPTION OF THE INVENTION

As described and illustrated herein, 3,11b-cis-dihydrotetrabenazine is useful for treatment of asthma. Accordingly, one aspect of the invention is a method for the prophylaxis or treatment of asthma in a patient, which method includes administering to the patient a therapeutically effective amount of a 3,11b cis-dihydrotetrabenazine, or an isomer or pharmaceutically acceptable salt thereof, to thereby treat asthma.

International patent application WO 2005/077946 (Cambridge Laboratories (Ireland) Limited), which is specifically incorporated herein in its entirety, discloses the preparation and pharmaceutical compositions of a group of 3,11b-cis-dihydrotetrabenazine isomers. WO 2007/017643 (Cambridge Laboratories (Ireland) Limited), which is specifically incorporated herein in its entirety, discloses the use of the 3,11b-cis-dihydrotetrabenazine isomers as anti-inflammatory agents.

The 3,11b-cis-dihydrotetrabenazine used in the methods and compositions of the invention may be in substantially pure form, for example, at an isomeric purity of greater than 90%, typically greater than 95% and more preferably greater than 98%.

The term “isomeric purity” in the present context refers to the amount of 3,11b-cis-dihydrotetrabenazine present relative to the total amount or concentration of dihydrotetrabenazine of all isomeric forms. For example, if 90% of the total dihydrotetrabenazine present in the composition is 3,11b-cis-dihydrotetrabenazine, then the isomeric purity is 90%.

The 3,11b-cis-dihydrotetrabenazine may be in the form of a composition which is substantially free of 3,11b-trans-dihydrotetrabenazine, for example, containing less than 5% of 3,11b-trans-dihydrotetrabenazine, or less than 3% of 3,11b-trans-dihydrotetrabenazine, or less than 1% of 3,11b-trans-dihydrotetrabenazine.

The term “3,11b-cis-” as used herein means that the hydrogen atoms at the 3- and 11b-positions of the dihydrotetrabenazine structure are in the cis orientation relative to each other. The isomers of the invention are therefore compounds of the formula (I) and antipodes (mirror images) thereof.

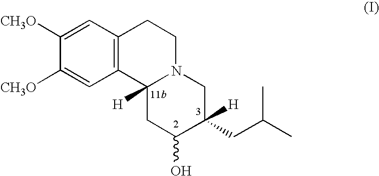

There are four possible isomers of dihydrotetrabenazine having the 3,11b-cis configuration and these are the 2S,3S,11bR isomer, the 2R,3R,11bS isomer, the 2R,3S,11bR isomer and the 2S,3R,11bS isomer. The four isomers have been isolated and characterised and, in another aspect, the invention provides individual isomers of 3,11b-cis-dihydrotetrabenazine for use in accordance with the invention. In particular, the invention provides the use, in the prophylaxis or treatment of asthma, of:

(a) the 2S,3S,11bR isomer of 3,11b-cis-dihydrotetrabenazine having the formula (Ia):

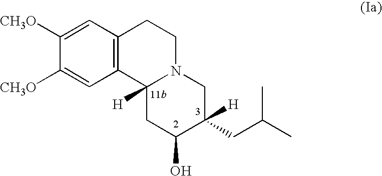

(b) the 2R,3R,11bS isomer of 3,11b-cis-dihydrotetrabenazine having the formula (Ib):

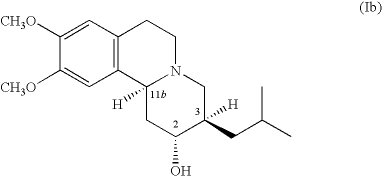

(c) the 2R,3S,11bR isomer of 3,11b-cis-dihydrotetrabenazine having the formula (Ic):

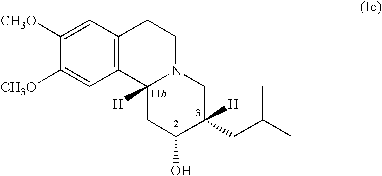

and

(d) the 2S,3R,11bS isomer of 3,11b-cis-dihydrotetrabenazine having the formula (Id):

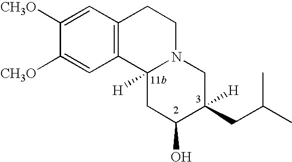

The individual isomers of the invention can be characterised by their spectroscopic, optical and chromatographic properties, and also by their absolute stereochemical configurations as determined by X-ray crystallography.

Without implying any particular absolute configuration or stereochemistry, the four 3,11b cis-dihydrotetrabenazine isomers may have the following characteristics.

The individual isomers of the invention can be characterised by their spectroscopic, optical and chromatographic properties, and also by their absolute stereochemical configurations as determined by X-ray crystallography.

For example, the four 3,11b cis-dihydrotetrabenazine isomers may be characterised as follows:

### Isomer A

Optical activity as measured by optical rotatory dispersion (ORD; methanol, 21° C.) as laevorotatory (−), infrared (IR) analysis (KBr solid), nuclear magnetic resonance (NMR), particularly ¹H-NMR (CDCl₃) and/or ¹³C-NMR (CDCl₃) analysis substantially as described in Table 1. Isomer A corresponds to formula (Ib) above.

### Isomer B

Optical activity as measured by ORD (methanol, 21° C.) as dextrorotatory (+), IR (KBr solid) analysis, NMR particularly ¹H-NMR (CDCl₃) and ¹³C-NMR (CDCl₃) analysis, substantially as described in Table 1, and X-ray crystallographic properties as described in Example 4. Isomer B corresponds to formula (Ia) above.

### Isomer C

Optical activity as measured by ORD (methanol, 21° C.) as dextrorotatory (+), IR (KBr solid) analysis, NMR particularly ¹H-NMR (CDCl₃) and ¹³C-NMR (CDCl₃) analysis, substantially as described in Table 2. Isomer C corresponds to either formula (Ic) or (Id) above.

### Isomer D

Optical activity as measured by ORD (methanol, 21° C.) as laevorotatory (−), IR (KBr solid) analysis, NMR particularly ¹H-NMR (CDCl₃) and ¹³C-NMR (CDCl₃) analysis, substantially as described in Table 2. Isomer D corresponds to either formula (Ic) or formula (Id) above.

ORD values for each isomer are given in the examples below but it is noted that such values are given by way of example and may vary according to the degree of purity of the isomer and the influence of other variables such as temperature fluctuations and the effects of residual solvent molecules.

The isomers A, B, C and D can be used in a substantially enantiomerically pure form or as mixtures with other 3,11b cis-dihydrotetrabenazine isomers.

The terms “enantiomeric purity” and “enantiomerically pure” in the present context refer to the amount of a given enantiomer of 3,11b-cis-dihydrotetrabenazine present relative to the total amount or concentration of dihydrotetrabenazine of all enantiomeric and isomeric forms. For example, if 90% of the total dihydrotetrabenazine present in the composition is in the form of a single enantiomer, then the enantiomeric purity is 90%.

By way of example, in each aspect and embodiment of the invention, each individual enantiomer selected from Isomers A, B, C and D may be present in an enantiomeric purity of at least 55% (e.g. at least 60%, 65%, 70%, 75%, 80%, 85%, 90%, 95%, 97%, 98%, 99%, 99.5% or 100%).

The isomers of the invention may also be presented in the form of mixtures of one or more of Isomers A, B, C and D. Such mixtures may be racemic mixtures or non-racemic mixtures. Examples of racemic mixtures include the racemic mixture of Isomer A and Isomer B and the racemic mixture of Isomer C and Isomer D.

### Pharmaceutically Acceptable Salts

Unless the context requires otherwise, a reference in this application to 3,11b-cis-dihydrotetrabenazine and its isomers includes within its scope not only the free base of the dihydrotetrabenazine but also its salts. One example of a pharmaceutically acceptable dihydrotetrabenazine salt is an acid addition salt of dihydrotetrabenazine.

Particular acids from which the acid addition salts are formed include acids having a pKa value of less than 3.5 and more usually less than 3. For example, the acid addition salts can be formed from an acid having a pKa in the range from +3.5 to −3.5.

Acid addition salts include those formed with sulphonic acids such as methanesulphonic acid, ethanesulphonic acid, benzene sulphonic acid, toluene sulphonic acid, camphor sulphonic acid and naphthalene sulphonic acid.

One acid from which acid addition salts may be formed is methanesulphonic acid.

Acid addition salts can be prepared by the methods described herein or conventional chemical methods such as the methods described in Pharmaceutical Salts: Properties, Selection, and Use, P. Heinrich Stahl (Editor), Camille G. Wermuth (Editor), ISBN: 3-90639-026-8, Hardcover, 388 pages, August 2002. Generally, such salts can be prepared by reacting the free base form of the compound with the appropriate base or acid in water or in an organic solvent, or in a mixture of the two. In some embodiments, a nonaqueous media such as ether, ethyl acetate, ethanol, isopropanol, or acetonitrile can be used.

The salts are typically pharmaceutically acceptable salts. However, salts that are not pharmaceutically acceptable may also be prepared as intermediate forms that may then be converted into pharmaceutically acceptable salts. Such non-pharmaceutically acceptable salt forms also form part of the invention.

Methods for the Preparation of 3,11b cis-dihydrotetrabenazine Isomers

The 3,11b cis-dihydrotetrabenazines of the invention can be prepared by the methods described in WO 2005/077946 and WO 2007/017643, which are specifically incorporated herein in their entireties, and as described in the examples below.

### Biological Activity

The 3,11b cis-dihydrotetrabenazine compounds of the invention have the ability to reduce the production of pro-inflammatory cytokines and inhibit T-cell proliferation as described in the Examples below. Beneficial activity has also been demonstrated in a chicken ovalbumin parenteral sensitization animal model of asthma. As such, the compounds of the invention are useful in preventing or treating asthma. For example, the compounds described herein are useful for treating a variety of asthma types, such as those where an infection, allergen, irritant, air-borne toxin, inflammatory agent or environment factor (e.g., cold, exercise) is a contributing factor to the asthma.

### Pharmaceutical Formulations

The 3,11b cis-dihydrotetrabenazine compounds can be administered in the form of pharmaceutical compositions.

The pharmaceutical compositions can be in any form suitable for oral, parenteral, topical, intranasal, intrabronchial, ophthalmic, otic, rectal, intra-vaginal, or transdermal administration. Where the compositions are intended for parenteral administration, they can be formulated for intravenous, intramuscular, intraperitoneal, subcutaneous administration or for direct delivery into a target organ or tissue by injection, infusion or other means of delivery.

Pharmaceutical dosage forms suitable for oral administration include tablets, capsules, caplets, pills, lozenges, syrups, solutions, sprays, powders, granules, elixirs and suspensions, sublingual tablets, sprays, wafers or patches and buccal patches.

Pharmaceutical compositions containing the dihydrotetrabenazine compounds of the invention can be formulated in accordance with known techniques, see, for example, Remington's Pharmaceutical Sciences, Mack Publishing Company, Easton, Pa., USA.

Thus, tablet compositions can contain a unit dosage of active compound together with an inert diluent or carrier such as a sugar or sugar alcohol, e.g.; lactose, sucrose, sorbitol or mannitol; and/or a non-sugar derived diluent such as sodium carbonate, calcium phosphate, talc, calcium carbonate, or a cellulose or derivative thereof such as methyl cellulose, ethyl cellulose, hydroxypropyl methyl cellulose, and starches such as corn starch. Tablets may also contain such standard ingredients as binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures. Such excipients are well known and do not need to be discussed in detail here.

Capsule formulations may be of the hard gelatin or soft gelatin variety and can contain the active component in solid, semi-solid, or liquid form. Gelatin capsules can be formed from animal gelatin or synthetic or plant derived equivalents thereof.

The solid dosage forms (e.g. tablets, capsules etc.) can be coated or un-coated, but typically have a coating, for example a protective film coating (e.g. a wax or varnish) or a release controlling coating. The coating (e.g. a Eudragit™ type polymer) can be designed to release the active component at a desired location within the gastro-intestinal tract. Thus, the coating can be selected so as to degrade under certain pH conditions within the gastrointestinal tract, thereby selectively release the compound in the stomach or in the ileum or duodenum.

Instead of, or in addition to, a coating, the drug can be presented in a solid matrix comprising a release controlling agent, for example a release delaying agent that may be adapted to selectively release the compound under conditions of varying acidity or alkalinity in the gastrointestinal tract. Alternatively, the matrix material or release retarding coating can take the form of an erodible polymer (e.g. a maleic anhydride polymer) which is substantially continuously eroded as the dosage form passes through the gastrointestinal tract.

Compositions for topical use include ointments, creams, sprays, patches, gels, liquid drops and inserts (for example intraocular inserts). Such compositions can be formulated in accordance with known methods.

Compositions for parenteral administration are typically presented as sterile aqueous or oily solutions or fine suspensions, or may be provided in finely divided sterile powder form for making up extemporaneously with sterile water for injection.

Examples of formulations for rectal or intra-vaginal administration include pessaries and suppositories which may be, for example, formed from a shaped mouldable or waxy material containing the active compound.

In some embodiments, the 3,11b cis-dihydrotetrabenazine compounds are presented as compositions for inhalation.

Compositions for administration by inhalation may take the form of inhalable powder compositions or liquid or powder sprays, and can be administrated in standard form using powder inhaler devices or aerosol dispensing devices. Such devices are well known. For administration by inhalation, the powdered formulations typically comprise the active compound together with an inert solid powdered diluent such as lactose or starch. Inhalable dry powder compositions may be presented in capsules and cartridges of gelatin or a like material, or blisters of laminated aluminum foil for use in an inhaler or insufflator. Each capsule or cartridge may generally contain between about 20 pg and about 10 mg of the active compound. Alternatively, the compound of the invention may be presented without excipients.

The inhalable compositions may be packaged for unit dose or multi-dose delivery. For example, the compositions can be packaged for multi-dose delivery in a manner analogous to that described in GB 2242134, U.S. Pat. No. 6,632,666, U.S. Pat. No. 5,860,419, U.S. Pat. No. 5,873,360 and U.S. Pat. No. 5,590,645 (all illustrating the “Diskus” device), or GB2178965, GB2129691, GB2169265, U.S. Pat. No. 4,778,054, U.S. Pat. No. 4,811,731 and U.S. Pat. No. 5,035,237 (which illustrate the “Diskhaler” device), or EP 69715 (“Turbuhaler” device), or GB 2064336 and U.S. Pat. No. 4,353,656 (“Rotahaler” device), the disclosures of which are specifically incorporated herein in their entireties.

Spray compositions for topical delivery to the lung by inhalation may be formulated as aqueous solutions or suspensions or as aerosols delivered from pressurized packs, such as a metered dose inhaler, with the use of a suitable liquefied propellant. Aerosol compositions suitable for inhalation can be presented either as suspensions or as solutions and typically contain the active compound and a suitable propellant such as a fluorocarbon or hydrogen-containing chlorofluorocarbon or mixtures thereof, particularly hydrofluoroalkanes such as dichlorodifluoromethane, trichlorofluoromethane, dichlorotetrafluoroethane, and especially 1,1, 1,2-tetrafluoroethane, 1,1, 1,2, 3,3, 3-heptafluoro-n-propane and mixtures thereof.

The aerosol composition may optionally contain additional excipients typically associated with such compositions, for example surfactants such as oleic acid or lecithin and cosolvents such as ethanol. Pressurized formulations can be contained within a canister (for example, an aluminum canister) closed with a metering valve and fitted into an actuator provided with a mouthpiece.

Medicaments for administration by inhalation desirably have a controlled particle size. The optimum particle size for inhalation into the bronchial system is usually 1-10 μm, preferably 2-5 μm. Particles having a size above 20 μm are generally too large when inhaled to reach the small airways. To achieve these particle sizes the particles of the active ingredient may be subjected to a size reducing process such as micronisation. The desired size fraction may be separated out by air classification or sieving. Preferably, the particles will be crystalline. When an excipient such as lactose is employed, typically the particle size of the excipient will be much greater than the particle size of the active ingredient.

Intranasal sprays may be formulated with aqueous or non-aqueous vehicles with the addition of agents such as thickening agents, buffer salts or acid or alkali to adjust the pH, isotonicity adjusting agents or anti-oxidants.

Solutions for inhalation by nebulization may be formulated with an aqueous vehicle with the addition of agents such as acid or alkali, buffer salts, isotonicity adjusting agents or antimicrobial agents. They may be sterilized by filtration or heating in an autoclave, or presented as a non-sterile product.

In some embodiments, the 3,11b cis-dihydrotetrabenazine is administered from a dry powder inhaler.

In other embodiments, the 3,11b cis-dihydrotetrabenazine is administered by an aerosol dispensing device, preferably in conjunction with an inhalation chamber such as the Volumatic™ inhalation chamber available from Allen & Hanbury, UK.

The compounds of the inventions will generally be presented in unit dosage form and, as such, will typically contain sufficient compound to provide a desired level of biological activity. For example, a formulation intended for oral administration may contain from 2 milligrams to 200 milligrams of active ingredient, more usually from 10 milligrams to 100 milligrams, for example, 12.5 milligrams, 25 milligrams and 50 milligrams.

The active compound will be administered to a patient in need thereof (for example a human or animal patient) in an amount sufficient to achieve the desired therapeutic effect.

The subject in need of such administration is a patient suffering from or at risk of suffering from an asthma attack.

The compounds will typically be administered in amounts that are therapeutically or prophylactically useful and which generally are non-toxic. However, in certain situations, particularly in the case of an acute life threatening asthma attack, the benefits of administering a dihydrotetrabenazine compound of the invention may outweigh the disadvantages of any toxic effects or side effects, in which case it may be considered desirable to administer the 3,11b cis-dihydrotetrabenazine in amounts that are associated with a degree of toxicity.

A typical daily dose of the compound can be up to 1000 mg per day, for example in the range from 0.01 milligrams to 10 milligrams per kilogram of body weight, more usually from 0.025 milligrams to 5 milligrams per kilogram of body weight, for example up to 3 milligrams per kilogram of bodyweight, and more typically 0.15 milligrams to 5 milligrams per kilogram of bodyweight although higher or lower doses may be administered where required.

### EXAMPLES

The following non-limiting examples illustrate the synthesis and properties of the 3,11b cis-dihydrotetrabenazine compounds of the invention.

### Example 1

### Preparation of 2S,3S,11bR and 2R,3R,11bS Isomers of Dihydrotetrabenazine

### 1A. Reduction of RR/SS Tetrabenazine

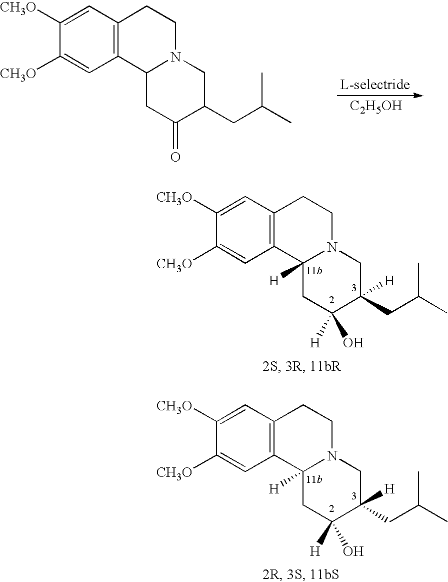

1M L-Selectride® in tetrahydrofuran (135 ml, 135 mmol, 2.87 eq.) was added slowly over 30 minutes to a stirred solution of tetrabenazine RR/SS racemate (15 g, 47 mmol) in ethanol (75 ml) and tetrahydrofuran (75 ml) at 0° C. After addition was complete the mixture was stirred at 0° C. for 30 minutes and then allowed to warm to room temperature.

The mixture was poured onto crushed ice (300 g) and water (100 ml) added. The solution was extracted with diethyl ether (2×200 ml) and the combined ethereal extracts washed with water (100 ml) and partly dried over anhydrous potassium carbonate. Drying was completed using anhydrous magnesium sulphate and, after filtration, the solvent was removed at reduced pressure (shielded from the light, bath temperature <20° C.) to afford a pale yellow solid.

The solid was slurried with petroleum ether (30-40° C.) and filtered to afford a white powdery solid (12 g, 80%).

### 1B. Dehydration of Reduced Tetrabenazine

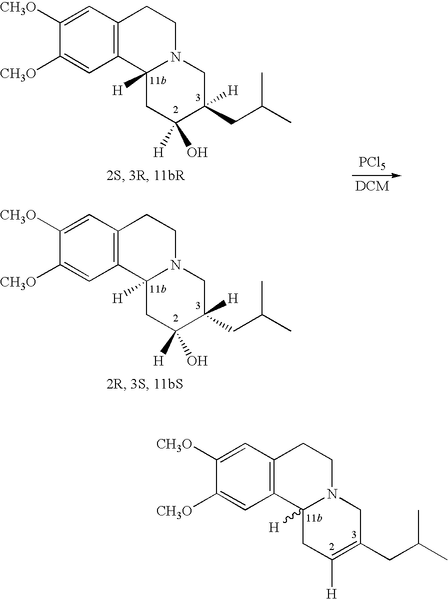

Phosphorous pentachloride (32.8 g, 157.5 mmol, 2.5 eq) was added in portions over 30 minutes to a stirred solution of the reduced tetrabenazine product from Example 1A (20 g, 62.7 mmol) in dichloromethane (200 ml) at 0° C. After the addition was complete, the reaction mixture was stirred at 0° C. for a further 30 minutes and the solution poured slowly into 2M aqueous sodium carbonate solution containing crushed ice (0° C.). Once the initial acid gas evolution had ceased the mixture was basified (ca. pH 12) using solid sodium carbonate.

The alkaline solution was extracted using ethyl acetate (800 ml) and the combined organic extracts dried over anhydrous magnesium sulphate. After filtration the solvent was removed at reduced pressure to afford a brown oil, which was purified by column chromatography (silica, ethyl acetate) to afford the semi-pure alkene as a yellow solid (10.87 g, 58%).

### 1C. Hydration of the Crude Alkene from Example 1B

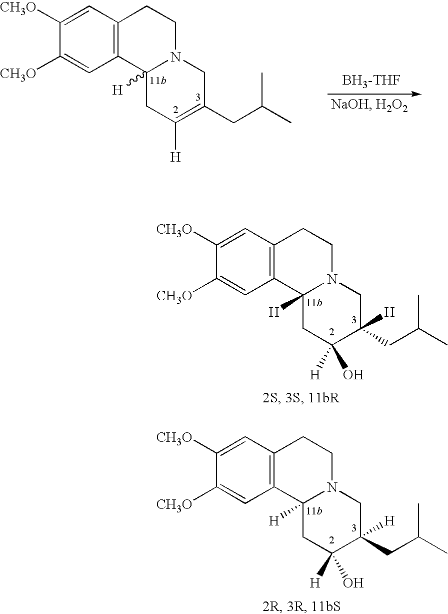

A solution of the crude alkene (10.87 g, 36.11 mmol) from Example 1B in dry THF (52 ml) at room temperature was treated with 1M borane-THF (155.6 ml, 155.6 mmol, 4.30 eq) added in a dropwise manner. The reaction was stirred for 2 hours, water (20 ml) was added and the solution basified to pH 12 with 30% aqueous sodium hydroxide solution.

Aqueous 30% hydrogen peroxide solution (30 ml) was added to the stirred alkaline reaction mixture and the solution was heated to reflux for 1 hour before being allowed to cool. Water (100 ml) was added and the mixture extracted with ethyl acetate (3×250 ml). The organic extracts were combined and dried over anhydrous magnesium sulphate and after filtration the solvent was removed at reduced pressure to afford a yellow oil (9 g).

The oil was purified using preparative HPLC (Column: Lichrospher Si60, 5 μm, 250×21.20 mm, mobile phase:hexane:ethanol:dichloromethane (85:15:5); UV 254 nm, flow: 10 ml min⁻¹) at 350 mg per injection followed by concentration of the fractions of interest under vacuum. The product oil was then dissolved in ether and concentrated once more under vacuum to give the dihydrotetrabenazine racemate shown above as a yellow foam (5.76 g, 50%).

### 1D. Preparation of Mosher's Ester Derivatives

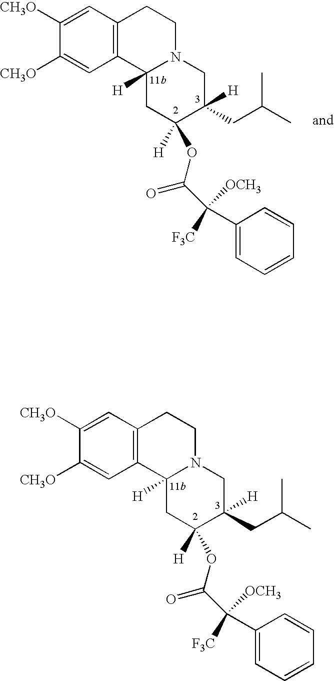

R-(+)-α-methoxy-α-trifluoromethylphenyl acetic acid (5 g, 21.35 mmol), oxalyl chloride (2.02 ml) and DMF (0.16 ml) were added to anhydrous dichloromethane (50 ml) and the solution was stirred at room temperature for 45 minutes. The solution was concentrated under reduced pressure and the residue was taken up in anhydrous dichloromethane (50 ml) once more. The resulting solution was cooled using an ice-water bath and dimethylaminopyridine (3.83 g, 31.34 mmol) was added followed by a pre-dried solution (over 4 Å sieves) in anhydrous dichloromethane of the solid product of Example 1C (5 g, 15.6 mmol). After stirring at room temperature for 45 minutes, water (234 ml) was added and the mixture extracted with ether (2×200 ml). The ether extract was dried over anhydrous magnesium sulphate, passed through a pad of silica and the product eluted using ether.

The collected ether eluate was concentrated under reduced pressure to afford an oil which was purified using column chromatography (silica, hexane:ether (10:1)). Evaporation of the collected column fractions of interest and removal of the solvent at reduced pressure gave a solid which was further purified using column chromatography (silica, hexane:ethyl acetate (1:1)) to give three main components which were partially resolved into Mosher's ester peaks 1 and 2.

Preparative HPLC of the three components (Column: 2×Lichrospher Si60, 5 μm, 250×21.20 mm, mobile phase:hexane:isopropanol (97:3), UV 254 nm; flow: 10 ml min⁻¹) at 300 mg loading followed by concentration of the fractions of interest under vacuum gave the pure Mosher's ester derivatives

Peak 1 (3.89 g, 46.5%)

Peak 2 (2.78 g, 33%)

The fractions corresponding to the two peaks were subjected to hydrolysis to liberate the individual dihydrotetrabenazine isomers identified and characterised as Isomers A and B. Isomers A and B are each believed to have one of the following structures

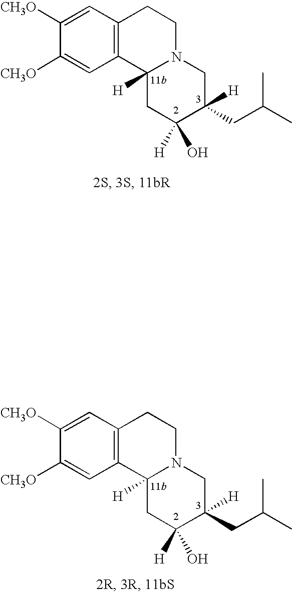

More specifically, Isomer B is believed to have the 2S, 3S, 11bR absolute configuration on the basis of the X-ray crystallography experiments described in Example 4 below.

### 1E. Hydrolysis of Peak 1 to give Isomer A

Aqueous 20% sodium hydroxide solution (87.5 ml) was added to a solution of Mosher's ester peak 1 (3.89 g, 7.27 mmol) in methanol (260 ml) and the mixture stirred and heated to reflux for 150 minutes. After cooling to room temperature water (200 ml) was added and the solution extracted with ether (600 ml), dried over anhydrous magnesium sulphate and after filtration, concentrated under reduced pressure.

The residue was dissolved using ethyl acetate (200 ml), the solution washed with water (2×50 ml), the organic phase dried over anhydrous magnesium sulphate and after filtration, concentrated under reduced pressure to give a yellow foam. This material was purified by column chromatography (silica, gradient elution of ethyl acetate:hexane (1:1) to ethyl acetate). The fractions of interest were combined and the solvent removed at reduced pressure. The residue was taken up in ether and the solvent removed at reduced pressure once more to give Isomer A as an off-white foam (1.1 g, 47%).

Isomer A, which is believed to have the 2R,3R,11bS configuration (the absolute stereochemistry was not determined), was characterized by ¹H-NMR, ¹³C-NMR, IR, mass spectrometry, chiral HPLC and ORD. The IR, NMR and MS data for isomer A are set out in Table 1 and the Chiral HPLC and ORD data are set out in Table 3.

### 1F. Hydrolysis of Peak 2 to Give Isomer B

Aqueous 20% sodium hydroxide solution (62.5 ml) was added to a solution of Mosher's ester peak 2 (2.78 g, 5.19 mmol) in methanol (185 ml) and the mixture stirred and heated to reflux for 150 minutes. After cooling to room temperature water (142 ml) was added and the solution extracted with ether (440 ml), dried over anhydrous magnesium sulphate and after filtration, concentrated under reduced pressure.

The residue was dissolved using ethyl acetate (200 ml), the solution washed with water (2×50 ml), the organic phase dried over anhydrous magnesium sulphate and after filtration, concentrated under reduced pressure. Petroleum ether (30-40° C.) was added to the residue and the solution concentrated under vacuum once more to give Isomer B as a white foam (1.34 g, 81%).

Isomer B, which is believed to have the 2S,3S,11bR configuration, was characterized by ¹H-NMR, ¹³C-NMR, IR, mass spectrometry, chiral HPLC, ORD and X-ray crystallography. The IR, NMR and MS data for Isomer B are set out in Table 1 and the Chiral HPLC and ORD data are set out in Table 3. The X-ray crystallography data are set out in Example 4.

### Example 2

### Preparation of 2R,3S,11bR and 2S,3R,11bS Isomers of Dihydrotetrabenazine

### 2A. Preparation of 2,3-Dehydrotetrabenazine

A solution containing a racemic mixture (15 g, 47 mmol) of RR and SS tetrabenazine enantiomers in tetrahydrofuran was subjected to reduction with L-Selectride® by the method of Example 1A to give a mixture of the 2S,3R,11bR and 2R,3S,11bS enantiomers of dihydrotetrabenazine as a white powdery solid (12 g, 80%). The partially purified dihydrotetrabenazine was then dehydrated using PCl₅ according to the method of Example 1B to give a semi-pure mixture of 11bR and 11bS isomers of 2,3-dehydrotetrabenazine (the 11bR enantiomer of which is shown below) as a yellow solid (12.92 g, 68%).

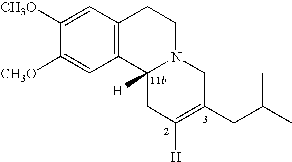

### 2B. Epoxidation of the Crude Alkene from Example 2A

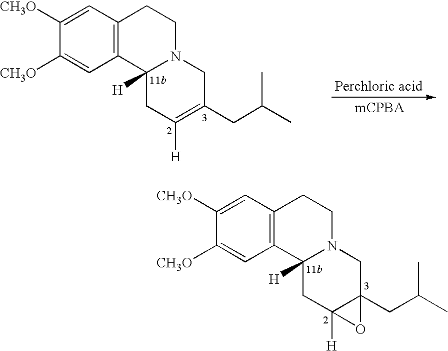

To a stirred solution of the crude alkene from Example 2A (12.92 g, 42.9 mmol) in methanol (215 ml) was added a solution of 70% perchloric acid (3.70 ml, 43 mmol) in methanol (215 ml). 77% 3-Chloroperoxybenzoic acid (15.50 g, 65 mmol) was added to the reaction and the resulting mixture was stirred for 18 hours at room temperature protected from light.

The reaction mixture was poured into saturated aqueous sodium sulphite solution (200 ml) and water (200 ml) added. Chloroform (300 ml) was added to the resulting emulsion and the mixture basified with saturated aqueous sodium bicarbonate (400 ml).

The organic layer was collected and the aqueous phase washed with additional chloroform (2×150 ml). The combined chloroform layers were dried over anhydrous magnesium sulphate and after filtration the solvent was removed at reduced pressure to give a brown oil (14.35 g, yield>100%−probable solvent remains in product). This material was used without further purification.

### 2C. Reductive Ring Opening of the Epoxide from 2B

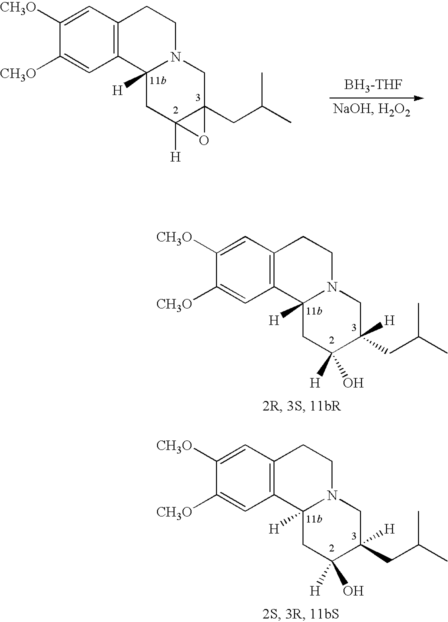

A stirred solution of the crude epoxide from Example 2B (14.35 g, 42 9 mmol, assuming 100% yield) in dry THF (80 ml) was treated slowly with 1M borane/THF (184.6 ml, 184.6 mmol) over 15 minutes. The reaction was stirred for two hours, water (65 ml) was added and the solution heated with stirring to reflux for 30 minutes.

After cooling, 30% sodium hydroxide solution (97 ml) was added to the reaction mixture followed by 30% hydrogen peroxide solution (48.6 ml) and the reaction was stirred and heated to reflux for an additional 1 hour.

The cooled reaction mixture was extracted with ethyl acetate (500 ml) dried over anhydrous magnesium sulphate and after filtration the solvent was removed at reduced pressure to give an oil. Hexane (230 ml) was added to the oil and the solution re-concentrated under reduced pressure.

The oily residue was purified by column chromatography (silica, ethyl acetate). The fractions of interest were combined and the solvent removed under reduced pressure. The residue was purified once more using column chromatography (silica, gradient, hexane to ether). The fractions of interest were combined and the solvents evaporated at reduced pressure to give a pale yellow solid (5.18 g, 38%).

### 2D. Preparation of Mosher's Ester Derivatives of the 2R,3S,11bR and 2S,3R,11bS Isomers of Dihydrotetrabenazine

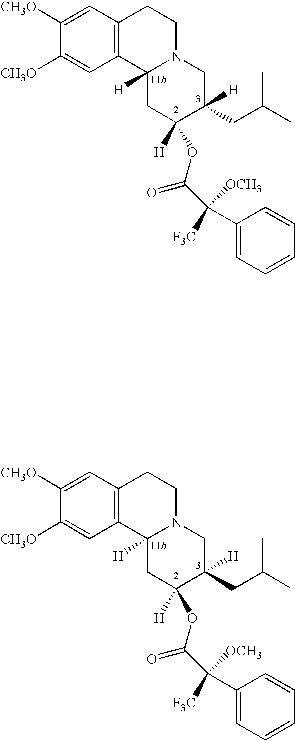

R-(+)-α-methoxy-α-trifluoromethylphenyl acetic acid (4.68 g, 19.98 mmol), oxalyl chloride (1.90 ml) and DMF (0.13 ml) were added to anhydrous dichloromethane (46 ml) and the solution stirred at room temperature for 45 minutes. The solution was concentrated under reduced pressure and the residue was taken up in anhydrous dichloromethane (40 ml) once more. The resulting solution was cooled using an ice-water bath and dimethylaminopyridine (3.65 g, 29.87 mmol) was added followed by a pre-dried solution (over 4 Å sieves) in anhydrous dichloromethane (20 ml) of the solid product of Example 2C (4.68 g, 14.6 mmol). After stirring at room temperature for 45 minutes, water (234 ml) was added and the mixture extracted with ether (2×200 ml). The ether extract was dried over anhydrous magnesium sulphate, passed through a pad of silica and the product eluted using ether.

The collected ether eluate was concentrated under reduced pressure to afford an oil which was purified using column chromatography (silica, hexane:ether (1:1)). Evaporation of the collected column fractions of interest and removal of the solvent at reduced pressure gave a pink solid (6.53 g)

Preparative HPLC of the solid (Column: 2×Lichrospher Si60, 5 μm, 250×21.20 mm; mobile phase hexane : isopropanol (97:3); UV 254 nm; flow: 10 ml min⁻¹) at 100 mg loading followed by concentration of the fractions of interest under vacuum gave a solid which was slurried with petroleum ether (30-40° C.) and collected by filtration to give the pure Mosher's ester derivatives

Peak 1 (2.37 g, 30%)

Peak 2 (2.42 g, 30%)

The fractions corresponding to the two peaks were subjected to hydrolysis to liberate the individual dihydrotetrabenazine isomers identified and characterised as Isomers C and D. Isomers C and D are each believed to have one of the following structures

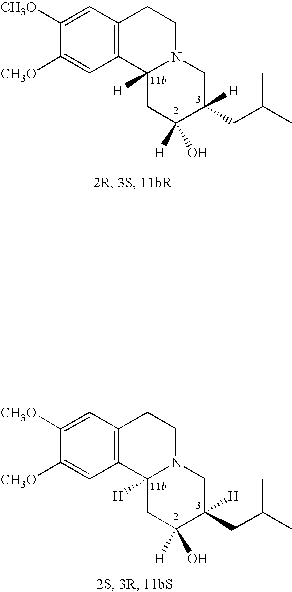

### 2F. Hydrolysis of Peak 1 to Give Isomer C

20% aqueous sodium hydroxide solution (53 ml) was added to a stirred solution of Mosher's ester peak 1 (2.37 g, 4.43 mmol) in methanol (158 ml) and the mixture stirred at reflux for 150 minutes. After cooling water (88 ml) was added to the reaction mixture and the resulting solution extracted with ether (576 ml). The organic extract was dried over anhydrous magnesium sulphate and after filtration the solvent removed at reduced pressure. Ethyl acetate (200 ml) was added to the residue and the solution washed with water (2×50 ml). The organic solution was dried over anhydrous magnesium sulphate and after filtration the solvent removed at reduced pressure.

This residue was treated with petroleum ether (30-40° C.) and the resulting suspended solid collected by filtration. The filtrate was concentrated at reduced pressure and the second batch of suspended solid was collected by filtration. Both collected solids were combined and dried under reduced pressure to give Isomer C (1.0 g, 70%).

Isomer C, which is believed to have either the 2R,3S,11bR or 2S,3R,11bS configuration (the absolute stereochemistry was not determined), was characterized by ¹H-NMR, ¹³C-NMR, IR, mass spectrometry, chiral HPLC and ORD. The IR, NMR and MS data for Isomer C are set out in Table 2 and the Chiral HPLC and ORD data are set out in Table 4.

### 2G. Hydrolysis of Peak 2 to Give Isomer D

20% aqueous sodium hydroxide solution (53 ml) was added to a stirred solution of Mosher's ester peak 2 (2.42 g, 4.52 mmol) in methanol (158 ml) and the mixture stirred at reflux for 150 minutes. After cooling water (88 ml) was added to the reaction mixture and the resulting solution extracted with ether (576 ml). The organic extract was dried over anhydrous magnesium sulphate and after filtration the solvent removed at reduced pressure. Ethyl acetate (200 ml) was added to the residue and the solution washed with water (2×50 ml). The organic solution was dried over anhydrous magnesium sulphate and after filtration the solvent removed at reduced pressure.

This residue was treated with petroleum ether (30-40° C.) and the resulting suspended orange solid collected by filtration. The solid was dissolved in ethyl acetate:hexane (15:85) and purified by column chromatography (silica, gradient ethyl acetate:hexane (15:85) to ethyl acetate). The fractions of interest were combined and the solvent removed at reduced pressure. The residue was slurried with petroleum ether (30-40° C.) and the resulting suspension collected by filtration. The collected solid was dried under reduced pressure to give Isomer D as a white solid (0.93 g, 64%).

Isomer D, which is believed to have either the 2R,3S,11bR or 2S,3R,11bS configuration (the absolute stereochemistry was not determined), was characterized by ¹H-NMR, ¹³C-NMR, IR, mass spectrometry, chiral HPLC and ORD. The IR, NMR and MS data for Isomer D are set out in Table 2 and the Chiral HPLC and ORD data are set out in Table 4.

In Tables 1 and 2, the infra red spectra were determined using the KBr disc method. The ¹H NMR spectra were carried out on solutions in deuterated chloroform using a Varian Gemini NMR spectrometer (200 MHz.). The ¹³C NMR spectra were carried out on solutions in deuterated chloroform using a Varian Gemini NMR spectrometer (50 MHz). The mass spectra were obtained using a Micromass Platform II (ES⁺ conditions) spectrometer. In Tables 3 and 4, the Optical Rotatory Dispersion figures were obtained using an Optical Activity PolAAr 2001 instrument in methanol solution at 24° C. The HPLC retention time measurements were carried out using an HP1050 HPLC chromatograph with UV detection.

### Tables 1 and 2—Spectroscopic Data

TABLE 1

| | ¹H-NMR | ¹³C-NMR | IR | Mass |
|---|---|---|---|---|
| | spectrum | spectrum | Spectrum | Spectrum |
| Dihydrotetrabenazine isomer | (CDCl₃) | (CDCl₃) | (KBr solid) | (ES⁺) |
| Isomers A and B | 6.67δ 1H (s); | 147.7δ; | 2950 cm⁻¹; | MH⁺ 320 |
| | 6.57 δ 1H (s); | 147.6δ; | 2928 cm⁻¹; | |
| | 3.84 δ 6H (s); | 130.5δ; | 2868 cm⁻¹; | |
| | 3.55 δ 1H (br. d); | 127.6δ; | 2834 cm⁻¹; | |
| 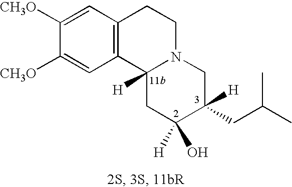 | 3.08 δ 1H (m); 2.79 δ 2H (m); 2.55 δ 3H (m); 2.17 δ 1H (m); | 112.1δ; 108.4δ; 70.5δ; 57.5 δl; | 1610 cm⁻¹; 1511 cm⁻¹; 1464 cm⁻¹; 1364 cm⁻¹; | |
| OR | | | | |
| 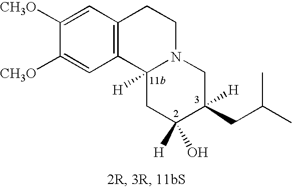 | 1.72 δ 6H (m); 1.02 δ 1H (m); 0.88 δ 6H (t) | 56.5 δ; 56.3 δ; 54.8 δ; 53.2 δ; 40.4 δ; 40.1 δ; 36.0 δ; 28.8 δ; 26.2 δ; 23.7δ; 22.9 δ | 1324 cm⁻¹; 1258 cm⁻¹; 1223 cm⁻¹; 1208 cm⁻¹; 1144 cm⁻¹; 1045 cm⁻¹; 1006 cm⁻¹; 870 cm⁻¹; 785 cm⁻¹; 764 cm⁻¹ | |

TABLE 2

| | ¹H-NMR | ¹³C-NMR | IR | Mass |
|---|---|---|---|---|
| | spectrum | spectrum | Spectrum | Spectrum |
| Dihydrotetrabenazine isomer | (CDCl₃) | (CDCl₃) | (KBr solid) | (ES⁺) |
| Isomers C and D | 6.58 δ 1H (s); | 147.8 δ; | 3370 cm⁻¹; | MH⁺ 320 |
| | 6.58 δ 1H (s); | 147.7 δ; | 2950 cm⁻¹; | |
| | 3.92 δ 1H (m); | 130.4 δ; | 2929 cm⁻¹; | |
| | 3.84 δ 6H (s); | 127.2 δ; | 1611 cm⁻¹; | |
| | 3.15 δ 1H (m); | 112.0 δ; | 1512 cm⁻¹; | |
| | 2.87 δ 3H (m); | 108.3 δ; | 1463 cm⁻¹; | |
| | 2.43 δ 4H (m); | 72.4 δ; | 1362 cm⁻¹; | |
| | 1.81 δ 1H (m); | 61.2 δ; | 1334 cm⁻¹; | |
| | 1.64 δ 4H (m); | 58.3 δ; | 1259 cm⁻¹; | |
| | 1.21 δ 1H (m); | 56.5 δ; | 1227 cm⁻¹; | |
| | 0.94 δ 3H (d); | 56.3 δ; | 1148 cm⁻¹; | |
| 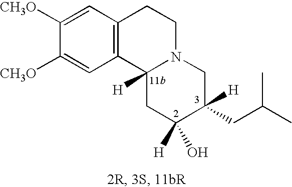 | 0.89 δ 3H (d) | 52.7 δ; 38.6 δ; 36.7 δ; 34.4 δ; 29.6 δ; | 1063 cm⁻¹; 1024 cm⁻¹; 855 cm⁻¹; 766 cm⁻¹ | |
| OR | | | | |
| 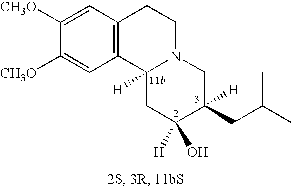 | | 26.5 δ; 24.4 δ; 22.5 δ | | |

### Tables 3 and 4—Chromatography and ORD Data

TABLE 3

| | | ORD |
|---|---|---|
| Dihydrotetrabenazine isomer | Chiral HPLC Methods and Retention Times | (MeOH, 21° C.) |
| Isomers A and B | Column: | Isomer A |
| 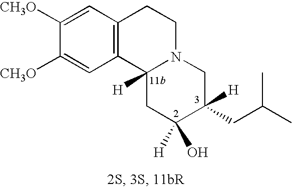 | Chirex (S)-VAL, (R)-NEA, 250 × 4.6 mm Mobile phase: Hexane: 1,2-dichloroethane ethanol (36:62:2) Flow: 1.0 ml min⁻¹ UV: 254 nm | [α_{D}] −114.6° Isomer B [α_{D}] +123° |
| OR | | |
| 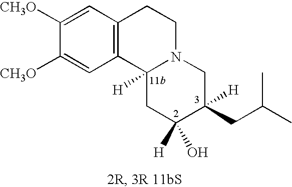 | Retention times: Isomer A 16.6 min Isomer B 15.3 min | |

TABLE 4

| Isomers C and D | Column: | | Isomer C |
|---|---|---|---|
| | Chirex (S)-VAL, (R)-NEA, 250 × 4.6 mm | | [α_{D}] +150.9° |
| 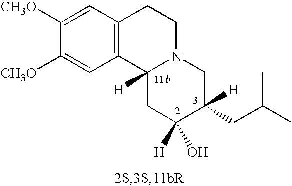 | Mobile phase: Flow: UV: | Hexane:ethanol (92:8) 1.0 ml min⁻¹ 254 nm | Isomer D [α_{D}] −145.7° |
| OR | | | |
| 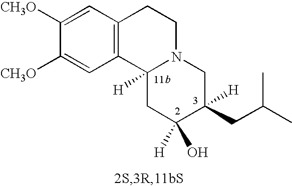 | Retention times: Isomer C Isomer D | 20.3 min 19.4 min | |

### Example 3

### Alternative Method of Preparation of Isomer B and Preparation of Mesylate Salt

### 3A. Reduction of RR/SS Tetrabenazine

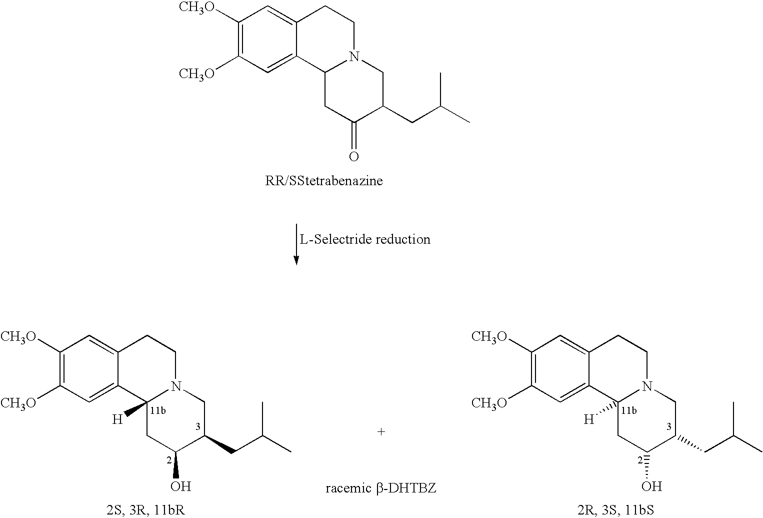

1M L-Selectride® in tetrahydrofuran (52 ml, 52 mmol, 1.1 eq) was added slowly over 30 minutes to a cooled (ice bath), stirred solution of tetrabenazine racemate (15 g, 47 mmol) in tetrahydrofuran (56 ml). After the addition was complete, the mixture was allowed to warm to room temperature and stirred for a further six hours. TLC analysis (silica, ethyl acetate) showed only very minor amounts of starting material remained.

The mixture was poured on to a stirred mixture of crushed ice (112 g), water (56 ml) and glacial acetic acid (12.2 g). The resulting yellow solution was washed with ether (2×50 ml) and basified by the slow addition of solid sodium carbonate (ca. 13 g). Pet-ether (30-40° C.) (56 ml) was added to the mixture with stirring and the crude β-DHTBZ was collected as a white solid by filtration.

The crude solid was dissolved in dichloromethane (ca. 150 ml) and the resulting solution washed with water (40 ml), dried using anhydrous magnesium sulphate, filtered and concentrated at reduced pressure to ca. 40 ml. A thick suspension of white solid was formed. Pet-ether (30-40° C.) (56 ml) was added and the suspension was stirred for fifteen minutes at laboratory temperature. The product was collected by filtration and washed on the filter until snow-white using pet-ether (30-40° C.) (40 to 60 ml) before air-drying at room temperature to yield β-DHTBZ (10.1 g, 67%) as a white solid. TLC analysis (silica, ethyl acetate) showed only one component.

### 3B. Preparation and Fractional Crystallisation of the Camphorsulphonic acid Salt of Racemic β-DHTBZ

The product of Example 3A and 1 equivalent of (S)-(+)-Camphor-10-sulphonic acid were dissolved with heating in the minimum amount of methanol. The resulting solution was allowed to cool and then diluted slowly with ether until formation of the resulting solid precipitation was complete. The resulting white crystalline solid was collected by filtration and washed with ether before drying.

The camphorsulphonic acid salt of (10 g) was dissolved in a mixture of hot absolute ethanol (170 ml) and methanol (30 ml). The resulting solution was stirred and allowed to cool. After two hours the precipitate formed was collected by filtration as a white crystalline solid (2.9 g). A sample of the crystalline material was shaken in a separating funnel with excess saturated aqueous sodium carbonate and dichloromethane. The organic phase was separated, dried over anhydrous magnesium sulphate, filtered and concentrated at reduced pressure. The residue was triturated using pet-ether (30-40° C.) and the organic solution concentrated once more. Chiral HPLC analysis of the salt using a Chirex (S)-VAL and (R)-NEA 250×4.6 mm column, and a hexane:ethanol (98:2) eluent at a flow rate of 1 ml/minute showed showed that the isolated β-DHTBZ was enriched in one enantiomer (e.e. ca. 80%).

The enriched camphorsulphonic acid salt (14 g) was dissolved in hot absolute ethanol (140 ml) and propan-2-ol (420 ml) was added. The resulting solution was stirred and a precipitate began to form within one minute. The mixture was allowed to cool to room temperature and stirred for one hour. The precipitate formed was collected by filtration, washed with ether and dried to give a white crystalline solid (12 g).

The crystalline material was shaken in a separating funnel with excess saturated aqueous sodium carbonate and dichloromethane. The organic phase was separated, dried over anhydrous magnesium sulphate, filtered and concentrated at reduced pressure. The residue was triturated using pet-ether (30-40° C.) and the organic solution concentrated once more to yield (after drying in vacuo.) (+)-β-DHTBZ (6.6 g, ORD+107.8°). The isolated enantiomer has e.e. >97%.

### 3C. Preparation of Isomer B

A solution of phosphorus pentachloride (4.5 g, 21.6 mmol, 1.05 eq) in dichloromethane (55 ml) was added steadily over ten minutes to a stirred, cooled (ice-water bath) solution of the product of Example 3B (6.6 g, 20.6 mmol) in dichloromethane (90 ml). When the addition was complete, the resulting yellow solution was stirred for a further ten minutes before pouring on to a rapidly stirred mixture of sodium carbonate (15 g) in water (90 ml) and crushed ice (90 g). The mixture was stirred for a further 10 minutes and transferred to a separating funnel.

Once the phases had separated, the brown dichloromethane layer was removed, dried over anhydrous magnesium sulphate, filtered and concentrated at reduced pressure to give the crude alkene intermediate as brown oil (ca. 6.7 g). TLC analysis (silica, ethyl acetate) showed that no (+)-β-DHTBZ remained in the crude product.

The crude alkene was taken up (dry nitrogen atmosphere) in anhydrous tetrahydrofuran (40 ml) and a solution of borane in THF (1 M solution, 2.5 eq, 52 ml) was added with stirring over fifteen minutes. The reaction mixture was then stirred at room temperature for two hours. TLC analysis (silica, ethyl acetate) showed that no alkene intermediate remained in the reaction mixture.

A solution of sodium hydroxide (3.7 g) in water (10 ml) was added to the stirring reaction mixture, followed by an aqueous solution of hydrogen peroxide (50%, ca. 7 ml) and the two-phase mixture formed was stirred at reflux for one hour. TLC analysis of the organic phase at this time (silica, ethyl acetate) showed the appearance of a product with Rf as expected for Isomer B. A characteristic non-polar component was also seen.

The reaction mixture was allowed to cool to room temperature and was poured into a separating funnel. The upper organic layer was removed and concentrated under reduced pressure to remove the majority of THF. The residue was taken up in ether (stabilised (BHT), 75 ml), washed with water (40 ml), dried over anhydrous magnesium sulphate, filtered and concentrated under reduced pressure to give a pale yellow oil (8.1 g).

The yellow oil was purified using column chromatography (silica, ethyl acetate:hexane (80:20), increasing to 100% ethyl acetate) and the desired column fractions collected, combined and concentrated at reduced pressure to give a pale oil which was treated with ether (stabilised, 18 ml) and concentrated at reduced pressure to give Isomer B as a pale yellow solid foam (2.2 g).

Chiral HPLC using the conditions set out in Example 3B confirmed that Isomer B had been produced in an enantiomeric excess (e.e.) of greater than 97%.

The optical rotation was measured using a Bellingham Stanley ADP220 polarimeter and gave an [α_{D}] of +123.5°.

### 3D. Preparation of the Mesylate Salt of Isomer B

The methanesulphonate salt of Isomer B was prepared by dissolving a mixture of 1 equivalent of Isomer B from Example 3C and 1 equivalent of methane sulphonic acid in the minimum amount of ethanol and then adding diethyl ether. The resulting white precipitate that formed was collected by filtration and dried in vacuo to give the mesylate salt in a yield of ca. 85% and a purity (by HPLC) of ca. 96%.

### Example 4

### X-Ray Crystallographic Studies on Isomer B

The (S)-(+)-Camphor-10-sulphonic acid salt of Isomer B was prepared and a single crystal was subjected to X-ray crystallographic studies under the following conditions:

Diffractometer: Nonius KappaCCD area detector (t/i scans and OJ scans to fill asymmetric unit).

Cell determination: DirAx (Duisenberg, A. J. M. (1992). J. Appl. Cryst. 25, 92-96.)

Data collection: Collect (Collect: Data collection software, R. Hooft, Nonius B. V, 1998)

Data reduction and cell refinement: Demo (Z. Otwinowski & W. Minor, Methods in Enzymology (1997) Vol. 276: Macromolecular Crystallography, part A, pp. 307-326; C. W. Carter, Jr & R. M. Sweet, Eds., Academic Press).

Absorption correction: Sheldrick, G. M. SADABS—Bruker Nonius area detector scaling and absorption correction—V 2.\0

Structure solution: SHELXS97 (G. M. Sheldrick, Acta Cryst. (1990) A46 467-473). Structure refinement: SHELXL97 (G. M. Sheldrick (1997), University of Göttingen, Germany)

Graphics: Cameron—A Molecular Graphics Package (D. M. Watkin, L. Pearce and C. K. Prout, Chemical Crystallography Laboratory, University of Oxford,1993)

Special details: All hydrogen atoms were placed in idealized positions and refined using a riding model, except those of the NH and OH which were located in the difference map and refined using restraints. Chirality: NI═R, CI2=S, CI3=S, CI5=R, C21=S, C24=R

The results of the studies are set out below in Tables A, B, C, D and E.

In the Tables, the label RUS0350 refers to Isomer B.

TABLE A

| Identification code | 2005bdy0585 (RUS0350) | |
|---|---|---|
| Empirical formula Formula weight Temperature Wavelength Crystal system Space group Unit cell dimensions Volume Z Density (calculated) Absorption coefficient F (000) Crystal Crystal size | C₂₉H₄₅NO₇S 551.72 120(2) K 0.71073 Å Orthorhombic P2₁2₁2₁ a = 7.1732 (9) Å b = 12.941 (2) Å c = 31.025 (4) Å 2880:1 (7) Å³ 4 1.272 Mg/m³ 0.158 mm⁻¹ 1192 Colourless Slab 0.2 × 0.2 × 0.04 mm³ | 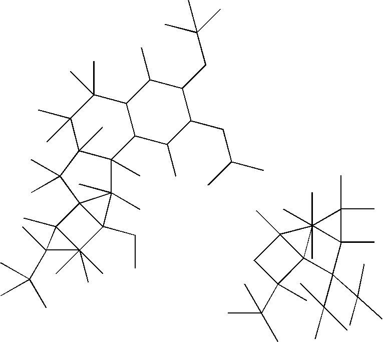 |

| | |
|---|---|
| θ range for data collection | 3.06 = 27.37° |
| Index ranges | −8 ≦ h ≦ 9, −16 ≦ k ≦ 16, −36 ≦/≦ 39 |
| Reflections collected | 36802 |
| Independent reflections | 6326 [Rₗₘ = 0.0863] |
| Completeness to θ =27.37° | 97.1% |
| Absorption correction | Semi-empirical from equivalents |
| Max. and min. transmission | 0.9937 and 0.9690 |
| Refinement method | Full-matrix least-squares on F² |
| Data/restraints/parameters | 6326/1/357 |
| Goodness-of-fit on F² | 1.042 |
| Final R indices [F² > 2σ(F²)] | R1 = 0.0498, wR2 = 0.0967 |
| R indices (all data) | R1 = 0.0901, wR2 = 0.1108 |
| Absolute structure parameter | 0.04 (8) |
| Extinction coefficient | 0.0059 (7) |
| Largest diff, peak and hole | 0.236 and −0.336 e Å⁻³ |

TABLE B
Atomic coordinates [×10⁴], equivalent isotropic displacement
parameters [A² × 10³] and site occupancy factors.

| Atom | x | y | z | Ueq | S.o.f. |
|---|---|---|---|---|---|
| NI | 4839(3) | 11119(2) | 2180(1) | 24(1) | 1 |
| 01 | 2515(3) | 13171(1) | 349(1) | 31(1) | 1 |
| 02 | 5581(3) | 14030(1) | 598(1) | 32(1) | 1 |
| 03 | 9220(3) | 12834(2) | 2385(1) | 36(1) | 1 |
| CI | 870(4) | 12674(2) | 190(1) | 36(1) | 1 |
| C2 | 3176(3) | 12838(2) | 739(1) | 25(1) | 1 |
| C3 | 2346(4) | 12109(2) | 997(1) | 25(1) | 1 |
| C4 | 3124(3) | 11821(2) | 1395(1) | 24(1) | 1 |
| C5 | 4773(3) | 12276(2) | 1527(1) | 23(1) | 1 |
| C6 | 5629(4) | 13024(2) | 1262(1) | 24(1) | 1 |
| C7 | 4861(4) | 13308(2) | 875(1) | 25(1) | 1 |
| C8 | 7189(4) | 14582(2) | 747(1) | 38(1) | 1 |
| C9 | 2182(3) | 11023(2) | 1673(1) | 28(1) | 1 |
| CI0 | 2759(3) | 11118(2) | 2137(1) | 26(1) | 1 |
| CII | 5366(3) | 11096(2) | 2656(1) | 25(1) | 1 |
| C12 | 7292(4) | 11536(2) | 2747(1) | 25(1) | 1 |
| C13 | 7468(4) | 12663(2) | 2590(1) | 25(1) | 1 |
| C14 | 5988(4) | 12911(2) | 2252(1) | 25(1) | 1 |
| C15 | 5773(4) | 12010(2) | 1943(1) | 24(1) | 1 |
| C16 | 7734(4) | 11477(2) | 3232(1) | 28(1) | 1 |
| C17 | 7752(4) | 10418(2) | 3449(1) | 34(1) | 1 |
| C18 | 9198(6) | 9696(3) | 3249(1) | 65(1) | 1 |
| C19 | 8114(4) | 10562(2) | 3930(1) | 41(1) | 1 |
| C20 | 7509(4) | 8131(2) | 1250(1) | 31(1) | 1 |
| S1 | 7409(1) | 8792(1) | 1754(1) | 27(1) | 1 |
| 04 | 7758(2) | 7965(1) | 2064(1) | 30(1) | 1 |
| 05 | 8831(3) | 9582(2) | 1760(1) | 49(1) | 1 |
| 06 | 5524(2) | 9221(1) | 1798(1) | 32(1) | 1 |
| 07 | 7406(3) | 6932(1) | 498(1) | 48(1) | 1 |
| C21 | 6858(3) | 8622(2) | 830(1) | 25(1) | 1 |
| C22 | 7154(4) | 7851(2) | 459(1) | 30(1) | 1 |
| C23 | 7073(4) | 8450(2) | 40(1) | 32(1) | 1 |
| C24 | 6648(3) | 9544(2) | 203(1) | 28(1) | 1 |
| C25 | 4742(3) | 8877(2) | 787(1) | 29(1) | 1 |
| C26 | 4742(3) | 8877(2) | 787(1) | 29(1) | 1 |
| C27 | 7773(4) | 9610(2) | 630(1) | 25(1) | 1 |
| C28 | 7431(4) | 10628(2) | 868(1) | 29(1) | 1 |
| C29 | 9895(4) | 9489(2) | 569(1) | 36(1) | 1 |

U_{eq} is defined as one third of the trace of the orthogonalized U^{ij} tensor.

TABLE C
Bond lengths [A] and angles [°].

| | | | |
|---|---|---|---|
| NI—CIO | 1.498(3) | C14—C15 | 1.518(3) |
| NI—CI5 | 1.522(3) | C16—C17 | 1.526(3) |
| NI—CII | 1.524(3) | C17—C18 | 1.527(4) |
| 01—C2 | 1.368(3) | C17—C19 | 1.527(4) |
| OI—CI | 1.432(3) | C20—C21 | 1.525(3) |
| 02—C7 | 1.369(3) | C20—SI | 1.784(2) |
| 02—C8 | 1.433(3) | SI—05 | 1.4442(19) |
| 03—C13 | 1.425(3) | SI—04 | 1.4607(17) |
| C2—C3 | 1.372(3) | SI—06 | 1.4676(18) |
| C2—C7 | 1.417(3) | 07—C22 | 1.208(3) |
| C3—C4 | 1.407(3) | C21—C22 | 1.537(4) |
| C4—C5 | 1.384(3) | C21—C26 | 1.559(3) |
| C4—C9 | 1.506(3) | C21—C27 | 1.565(3) |
| C5—C6 | 1.411(3) | C22—C23 | 1.517(4) |
| C5—C15 | 1.516(3) | C23—C24 | 1.535(4) |
| C6—C7 | 1.372(3) | C24—C25 | 1.548(4) |
| C9—CI0 | 1.504(3) | C24—C27 | 1.554(4) |
| CII—CI2 | 1.521(3) | C25—C26 | 1.557(4) |
| C12—C16 | 1.540(3) | C27—C28 | 1.529(3) |
| C12—C13 | 1.544(3) | C27—C29 | 1.542(4) |
| C13—C14 | 1.524(3) | | |
| CI0—NI—CI5 | 113.33(19) | CI2—CII—NI | 113.43(19) |
| CI0—NI—CII | 109.46(18) | CII—CI2—CI6 | 110.5(2) |
| CI5—NI—CII | 111.96(19) | CII—CI2—CI3 | 111.7(2) |
| C2—01—CI | 116.6(2) | CI6—CI2—CI3 | 109.84(19) |
| C7—02—C8 | 116.27(19) | 03—CI3—CI4 | 106.0(2) |
| 01—C2—C3 | 125.5(2) | 03—CI3—CI2 | 111.1(2) |
| 01—C2—C7 | 115.0(2) | CI4—CI3—CI2 | 111.0(2) |
| C3—C2—C7 | 119.5(2) | CI5—CI4—CI3 | 110.1(2) |
| C2—C3—C4 | 121.5(2) | C5—CI5—CI4 | 114.3(2) |
| C5—C4—C3 | 119.2(2) | C5—CI5—NI | 112.0(2) |
| C5—C4—C9 | 120.3(2) | CI4—CI5—NI | 108.7(2) |
| C3—C4—C9 | 120.5(2) | CI7—CI6—CI2 | 118.4(2) |
| C4—C5—C6 | 119.4(2) | CI6—CI7—CI8 | 112.2(2) |
| C4—C5—CI5 | 124.1(2) | CI6—CI7—CI9 | 108.7(2) |
| C6—C5—CI5 | 116.6(2) | CI8—CI7—CI9 | 110.8(3) |
| C7—C6—C5 | 121.3(2) | C21—C20—S1 | 122.51(18) |
| 02—C7—C6 | 125.4(2) | 05—SI—04 | 112.93(11) |
| 02—C7—C2 | 115.4(2) | 05—SI—06 | 112.47(12) |
| C6—C7—C2 | 119.2(2) | 04—SI—06 | 111.93(11) |
| CI0—C9—C4 | 111.7(2) | 05—SI—C20 | 108.81(13) |
| NI—CI0—09 | 111.0(2) | 04—SI—C20 | 102.60(11) |
| 06—SI—C20 | 107.44(12) | C23—C24—C25 | 106.4(2) |
| C20—C21—C22 | 109.0(2) | C23—C24—C27 | 103.3(2) |
| C20—C21—C26 | 117.3(2) | C25—C24—C27 | 102.3(2) |
| C22—C21—C26 | 102.1(2) | C24—C25—C26 | 102.9(2) |
| C20—C21—C27 | 123.4(2) | C25—C26—C21 | 104.2(2) |
| C22—C21—C27 | 100.21(19) | C28—C27—C29 | 107.8(2) |
| C26—C21—C27 | 101.7(2) | C28—C27—C24 | 112.0(2) |
| 07—C22—C23 | 126.4(2) | C29—C27—C24 | 113.7(2) |
| 07—C22—C21 | 125.9(2) | C28—C27—C21 | 116.5(2) |
| C23—C22—C21 | 107.7(2) | C29—C27—C21 | 112.3(2) |
| C22—C23—C24 | 101.3(2) | C24—C27—C21 | 94.27(19) |

TABLE D
Anisotropic displacement parameters [A² × 10³].
The anisotropic displacement factor exponent takes the form:-
2π²[h²a*²U¹¹ + . . . + 2 h k a* b* U^{I2}].

| Atom | U^{II} | U²² | U³³ | U²³ | U^{I3} | U¹² |
|---|---|---|---|---|---|---|
| NI | 26(1) | 24(1) | 23(1) | 2(1) | −1(1) | −3(1) |
| 01 | 37(1) | 30(1) | 24(1) | 3(1) | −7(1) | −4(1) |
| 02 | 41(1) | 31(1) | 25(1) | 5(1) | −2(1) | −10(1) |
| 03 | 26(1) | 49(1) | 32(1) | 7(1) | −3(1) | −9(1) |
| CI | 41(2) | 36(2) | 32(2) | 3(1) | −9(1) | −8(2) |
| C2 | 30(2) | 24(2) | 22(1) | 1(1) | −1(1) | 2(1) |
| C3 | 25(1) | 26(1) | 24(1) | −3(1) | −2(1) | 2(1) |
| C4 | 26(2) | 22(1) | 23(1) | −1(1) | 2(1) | −1(1) |
| C5 | 24(1) | 22(1) | 23(1) | −2(1) | 1(1) | 0(1) |
| C6 | 26(1) | 22(1) | 24(1) | −3(1) | 2(1) | −5(1) |
| C7 | 30(2) | 22(1) | 22(1) | 2(1) | 4(1) | −4(1) |
| C8 | 45(2) | 34(2) | 36(2) | 5(1) | −2(1) | −20(2) |
| C9 | 23(1) | 32(1) | 29(2) | 3(1) | −1(1) | −4(1) |
| CIO | 26(1) | 29(1) | 25(1) | 2(1) | 0(1) | −5(1) |
| C11 | 31(1) | 25(1) | 20(1) | 2(1) | 0(1) | −2(1) |
| C12 | 26(1) | 26(1) | 23(1) | −1(1) | 1(1) | −1(1) |
| CI3 | 26(1) | 28(1) | 23(1) | −1(1) | −1(1) | −2(1) |
| CI4 | 30(2) | 22(2) | 24(1) | −1(1) | 1(1) | −1(1) |
| CI5 | 22(1) | 22(1) | 28(1) | 2(1) | 0(1) | −4(1) |
| C16 | 31(1) | 28(1) | 24(1) | −1(1) | −3(1) | 3(1) |
| CI7 | 46(2) | 31(2) | 25(1) | 1(1) | −7(1) | 0(2) |
| CI8 | 106(3) | 46(2) | 41(2) | 6(2) | −1(2) | 31(2) |
| C19 | 51(2) | 41(2) | 31(2) | 9(2) | −7(1) | −4(2) |
| C20 | 30(2) | 34(2) | 29(1) | 2(1) | 3(1) | 9(2) |
| S1 | 27(1) | 30(1) | 24(1) | 4(1) | −2(1) | −5(1) |
| O4 | 31(1) | 36(1) | 23(1) | 9(1) | −1(1) | 0(1) |
| O5 | 53(1) | 58(1) | 37(1) | 13(1) | −11(1) | −35(1) |
| O6 | 34(1) | 35(1) | 28(1) | −3(1) | −2(1) | 10(1) |
| O7 | 81(2) | 25(1) | 40(1) | −1(1) | 12(1) | 6(1) |
| C21 | 26(1) | 25(2) | 24(1) | −1(1) | 3(1) | 2(1) |
| C22 | 35(2) | 25(2) | 31(2) | 0(1) | 1(1) | −1(1) |
| C23 | 40(2) | 30(2) | 25(1) | −2(1) | 1(1) | −2(1) |
| C24 | 28(1) | 29(2) | 26(2) | 2(1) | 2(1) | 2(1) |
| C25 | 30(2) | 34(2) | 29(2) | −1(1) | −2(1) | 0(1) |
| C26 | 26(1) | 34(2) | 28(2) | 0(1) | 1(1) | −5(1) |
| C27 | 23(1) | 26(1) | 26(1) | 0(1) | 2(1) | 0(1) |
| C28 | 31(1) | 26(1) | 30(1) | 0(1) | −2(1) | −6(1) |
| C29 | 29(2) | 41(2) | 40(2) | 0(2) | 2(1) | −3(1) |

TABLE E
Hydrogen coordinates [×10⁴] and isotropic displacement
parameters [Å² × 10³].

| Atom | x | y | z | U_{eq} | S.o.f |
|---|---|---|---|---|---|
| H98 | 5190(40) | 10528(15) | 2062(10) | 70(8) | 1 |
| H99 | 10030(50) | 12950(30) | 2575(12) | 70(8) | 1 |
| H1A | 1107 | 11933 | 156 | 54 | 1 |
| H1B | 529 | 12973 | −89 | 54 | 1 |
| H1C | −154 | 12777 | 395 | 54 | 1 |
| H3 | 1220 | 11793 | 904 | 30 | 1 |
| H6 | 6760 | 13337 | 1353 | 29 | 1 |
| H8A | 6872 | 14966 | 1009 | 58 | 1 |
| H8B | 7600 | 15065 | 523 | 58 | 1 |
| H8C | 8193 | 14091 | 810 | 58 | 1 |
| H9A | 814 | 11106 | 1651 | 33 | 1 |
| H9B | 2505 | 10324 | 1567 | 33 | 1 |
| H10A | 2250 | 11767 | 2259 | 32 | 1 |
| H10B | 2235 | 10534 | 2304 | 32 | 1 |
| H11A | 4431 | 11494 | 2822 | 30 | 1 |
| H11B | 5322 | 10372 | 2759 | 30 | 1 |
| H12 | 8230 | 11108 | 2589 | 30 | 1 |
| H13 | 7334 | 13145 | 2840 | 30 | 1 |
| H14A | 4783 | 13050 | 2397 | 30 | 1 |
| H14B | 6354 | 13538 | 2090 | 30 | 1 |
| H15 | 7056 | 11776 | 1864 | 29 | 1 |
| H16A | 8973 | 11796 | 3278 | 33 | 1 |
| H16B | 6813 | 11911 | 3386 | 33 | 1 |
| I H17 | 6493 | 10098 | 3412 | 41 | 1 |
| H18A | 8906 | 9588 | 2944 | 97 | 1 |
| H18B | 9176 | 9031 | 3400 | 97 | 1 |
| H18C | 10440 | 10005 | 3276 | 97 | 1 |
| H19A | 9329 | 10894 | 3971 | 62 | 1 |
| H19B | 8110 | 9887 | 4073 | 62 | 1 |
| H19C | 7135 | 10999 | 4054 | 62 | 1 |
| H20A | 8824 | 7924 | 1207 | 37 | 1 |
| H20B | 6787 | 7484 | 1286 | 37 | 1 |
| H23A | 6070 | 8190 | −151 | 38 | 1 |
| H23B | 8277 | 8423 | −116 | 38 | 1 |
| H24 | 6928 | 10107 | −8 | 33 | 1 |
| H25A | 3773 | 9195 | 153 | 37 | 1 |
| H25B | 4152 | 10235 | 426 | 37 | 1 |
| H26A | 3994 | 8237 | 764 | 35 | 1 |
| H26B | 4300 | 9279 | 1039 | 35 | 1 |
| H28A | 8160 | 10638 | 1135 | 44 | 1 |
| I H28B | 6103 | 10692 | 936 | 44 | 1 |
| H28C | 7811 | 11207 | 684 | 44 | 1 |
| H29A | 10358 | 10042 | 381 | 54 | 1 |
| H29B | 10159 | 8817 | 436 | 54 | 1 |
| H29C | 10517 | 9531 | 849 | 54 | 1 |

TABLE 6
Hydrogen bonds [Åand °].

| D—H···A | d(D—H) | d(H···A) | d(D···A) | ∠(DHA) |
|---|---|---|---|---|
| N1—H98···O6 | 0.885(10) | 1.895(12) | 2.773(3) | 171(3) |
| N1—H98···S1 | 0.885(10) | 2.914(14) | 3.771(2) | 163(3) |
| O3—H99···O4ⁱ | 0.84(4) | 1.94(4) | 2.766(3) | 165(3) |
| O3—H99···S1ⁱ | 0.84(4) | 2.98(4) | 3.811(2) | 169(3) |

Symmetry transformations used to generate equivalent atoms:
ⁱ−x + 2, y + ½, −z + ½

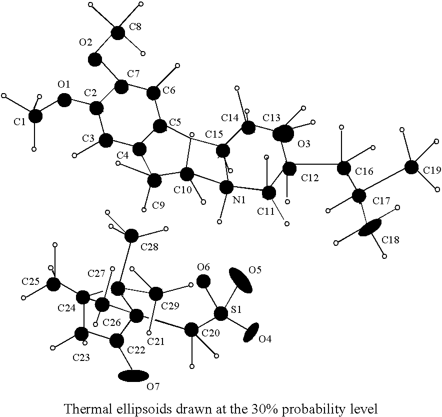

On the basis of the data set out above, Isomer B is believed to have the 2S, 3S, 11bR configuration, which corresponds to Formula (Ia):

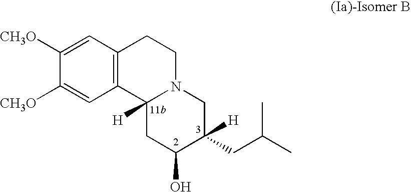

Isomer A, by elimination, therefore has the 2R, 3R, 11bS configuration, which corresponds to Formula (Ib):

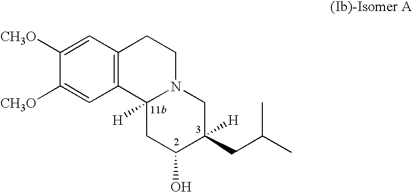

### Example 5

### A Study of the Effect of Isomer B (RU350) in a Chicken Ovalbumin Parenteral Sensitization Model of Asthma

An animal model of asthma was used in this study that included parenteral sensitisation of mice with chicken ovalbumin (OVA) together with a suitable adjuvant (Alum). Ovalbumin is widely used as an antigen as a result of its availability and ability to induce a good Th2-type immune response due to lack of any previous exposure to this antigen. Repeated aerosol exposure to ovalbumin post-sensitisation triggers airway changes leading to hyper-responsiveness, similar to that seen in asthma. These changes can be measured following challenge with a bronchoconstricting agent such as methacholine and analysed using whole body plethysmography. Pulmonary plethysmography is used to measure the functional residual capacity (FRC) of the lungs—the volume in the lungs when the muscles of respiration are relaxed—and total lung capacity. The degree of bronchochonstriction (BHR) can be expressed as enhanced pause (Pen H), a calculated value which correlates with measurement of airway resistance, impedance and intrapleural pressure in the same mouse.

Pen H is calculated from the relationship Pen H=(Te/Tr−1)×(Pef/Pif) where;

Te=expiration time

Tr=relaxation time

Pef=peak expiratory flow

Pif=peak inspiratory flow×0.67 coefficient

In addition, allergy can by analysed by examination of changes in the lung and lung fluid. This can be achieved by histopathological analysis of lung tissue and analysis of the cellular infiltrate in bronchial lavage fluid (BAL). Further, additional markers of allergy such as the presence of cytokines associated with allergy, IL-4 and IL-13 can be analysed in the BAL fluid.

### METHODS

### 3.1 Component 1: Clinical Endpoints

Groups of 8 BALB/c mice aged between 5 and 8 weeks of age were sensitised by i.p. (intraperitoneal) injection with ovalalbumin (OVA) in alum on days 0 and 14 (except Group A). All animals were challenged by aerosol exposure to 5% OVA for 20 minutes daily from days 18 to 23. Treatments were given by oral gavage twice daily from day 14 to day 24. At termination (day 24), all animals were subjected to unrestrained whole body plethysmography (whole-body plethysmograph Buxco Electronics, Troy, US) during exposure to increasing doses of methacholine leading to bronchoconstriction and hyper-responsiveness. These changes can be measured using the Buxco software to determine the PenH values for each animal. BAL fluids were collected and cytospins prepared and counted differentially for the presence of infiltrating cells. The supernatant from the BAL was retained and stored at ⁻80° C. for possible cytokine analysis. Further, lungs were removed and placed in 10% buffered formalin for possible histopathology.

Six groups of animals (n=8/group) were established, as follows:

A) Unsensitized/challenged/untreated

B) Sensitised/challenged/Untreated

C) Sensitised/challenged/treated RU350 1 mg/kg day 14-24 twice daily by oral gavage

D) Sensitised/challenged/treated with RU350 10 mg/kg day 14-24 twice daily by oral gavage

E) Sensitised/challenged/treated with RU350 20 mg/kg day 14-24 twice daily by oral gavage

F) Sensitised/challenged/treated with Budesonide 1 mg/kg day 14-24 twice daily by oral gavage

On day 0, mice in groups B-F were sensitised to ovalbumin by intra-peritoneal (i.p.) administration of 200 μl OVA/Alum (10 μg OVA). On day 14 the procedure was repeated. Group A remained unsensitized.

Treatments were delivered by oral gavage (100 μl per dose) twice daily from days 14 to 24 at appropriate concentrations as described above.

All mice were exposed to an OVA challenge (5% OVA in PBS) delivered by nebuliser for 20 minutes daily from day 18-23.

At termination (day 24), the animals in groups C-F were given the final treatment. All animals were exposed to increased concentrations of methacholine from 6.25 mg/ml to 100 mg/ml in PBS for measurement of unrestrained whole body plethysmography (PenH values).

Mice were terminated by i.p. injection of euthatal, the trachea exposed and cells obtained from the lungs by performing bronchoalveolar lavage, 3×0.4 ml with PBS. The lavage was pooled, cells counted using a nucleocounter, pelletted and resuspended at 5×10⁵ cells per ml. An aliquot of 100 μl was placed in a Cytospin™ centrifuge and spun onto a poly-1-lysine coated slide. Each sample was dried overnight and then stained with Leishmans for analysis of differential cell counts. The supernatant was retained for possible cytokine analysis.

Lungs were removed at termination and stored in 10% buffered formalin for histopathological analysis (Component 2).

### 3.2 Differential Cell Counts

Cells were viewed at ×100 oil immersion

Neutrophils Dark purple nuclei, pale pink cytoplasm, small purple granules

Eosinophils Blue nuclei, pale pink cytoplasm, large red/pink granules

Lymphocytes Purple nuclei, sky blue cytoplasm

Monocytes/macrophages Dark blue multi-lobed nuclei

A minimum of 5 areas were counted on each slide. Each cell type was counted and percentage cell numbers determined From this the number of cells/BAL was determined.

### 3.3 Histopathology Scores

### Samples:

Formalin-fixed, lung lobes from mice in 6 experimental groups A-F. Each lung from one mouse was assigned a Pathology numerical code (e.g. R0066-08).

### Methods:

For each lung, three standard sections were taken from three lobes (left and right caudal, right cranial). The samples were routinely processed, sectioned and one HE-stained section prepared for examination. The HE-stained sections were assessed for lung inflammation. Each lung was scored as described below. Samples were scored in blinded fashion, without knowledge of the experimental protocol or identity of groups.

### Scoring System:

### Pulmonary Inflammation

A semi-quantitative grading system was used to describe the degree of inflammatory change in the lungs. Descriptive comments (nature of cellular infiltration) were also recorded.

0 normal

1 low numbers of individual inflammatory cells around most airways and blood vessels

2 focal aggregates [more than 5 cells thick] of inflammatory cells adjacent to some airways and blood vessels

3 focal aggregates [more than 5 cells thick] of inflammatory cells adjacent to most airways and blood vessels

4 ‘cuffing’ of some airways and blood vessels by inflammatory cells [more than 5 cells thick]

5 ‘cuffing’ of most airways and blood vessels by inflammatory cells [more than 5 cells thick]

### 5. Conclusions

Unsensitized animals (Group A) had very few cells in the BAL fluid and showed only a minimal response to exposure to the bronchoconstricting agent, methacholine. In contrast, sensitisation and aerosol OVA challenge established a severe inflammatory reaction in the lungs, as evidenced by the high numbers of cells infiltrating the BAL and the much enhanced sensitivity to methacholine (Group B). As expected in this model, the inflammatory infiltrate was dominated by eosinophils, the major infiltrating cell within the human asthmatic lung. Treatment with the steroid, budesonide, markedly suppressed lung cell infiltration and reduced the airway hyper-responsiveness to methacholine to near control levels. Taken together, these data indicate that the experiment fell within expected parameters for studies using this model.

Three doses of RU350 (Isomer B) were used in this study. There was a dose dependent effect of RU350 on both levels of eosinophil infiltration into the lung and on the PenH response to methacholine. At the two lower doses tested there was no clear difference between treated and untreated animals but at the highest dose (Group E) RU350 reduced airway hyper-responsiveness to levels just above those of the budesonide controls, and there was an associated reduction in eosinophil numbers in the BAL. However, the effect on lung cell infiltration was small.

Further analysis of the mean group histopathological scores correlated well with the other observations. The unchallenged mice had essentially normal lungs, maximum severity of pathology was present in group B, and this is ameliorated by the positive control treatment (group F, P<0.01). There is also significant amelioration of pathology by the test agent Isomer B, and this has a probable dose-effect with reduced pathology score as the dose increases from 1 to 20 mg/kg (P<0.05 for group C and <0.01 for group D and E). The effects of RU350 were most marked in the pathological analysis.

### Example 6

### Pharmaceutical Compositions

### (i) Tablet Formulation—I

A tablet composition containing a dihydrotetrabenazine of the invention is prepared by mixing 50 mg of the dihydrotetrabenazine with 197 mg of lactose (BP) as diluent, and 3 mg magnesium stearate as a lubricant and compressing to form a tablet in known manner.

### (ii) Tablet Formulation—II

A tablet composition containing a dihydrotetrabenazine of the invention is prepared by mixing the compound (25 mg) with iron oxide, lactose, magnesium stearate, starch maize white and talc, and compressing to form a tablet in known manner.

(iii) Capsule Formulation

A capsule formulation is prepared by mixing 100 mg of a dihydrotetrabenazine of the invention with 100 mg lactose and filling the resulting mixture into standard opaque hard gelatin capsules.

### Equivalents

It will readily be apparent that numerous modifications and alterations may be made to the specific embodiments of the invention described above without departing from the principles underlying the invention. All such modifications and alterations are intended to be embraced by this application.

What is claimed:

1. A method for the prophylaxis or treatment of asthma in a patient, which method comprises administering to the patient a therapeutically effective amount of a 3,11b cis-dihydrotetrabenazine, or a pharmaceutically acceptable salt thereof, to thereby treat asthma.

2. The method of claim 1, wherein the asthma is of any one or more of asthma types selected from the group consisting of allergic asthma, non-allergic asthma, late onset asthma, early-onset asthma, exercise-induced asthma, nocturnal asthma, cough variant asthma, work-related asthma, work aggravated asthma, occupational asthma, asthma induced by large molecular weight compounds, IgE-dependent asthma, asthma induced by low molecular weight compounds, IgE-independent asthma, reactive airways dysfunction syndrome, and inner city or urban asthma.

3. The method of claim 1, wherein the asthma is allergic asthma or exercise-induced asthma.

4. The method of claim 1, wherein the 3,11b-cis-dihydrotetrabenazine, or a pharmaceutically acceptable salt thereof, is a 2S,3S,11bR isomer of 3,11b-cis-dihydrotetrabenazine having the formula (Ia): 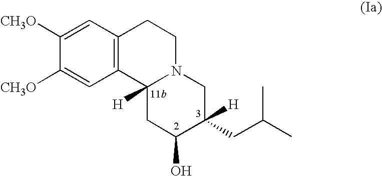
or a pharmaceutically acceptable salt thereof.

5. The method of claim 1, wherein the 3,11b-cis-dihydrotetrabenazine, or a pharmaceutically acceptable salt thereof, is a 2R,3R,11bS isomer of 3,11b-cis-dihydrotetrabenazine having the formula (Ib): 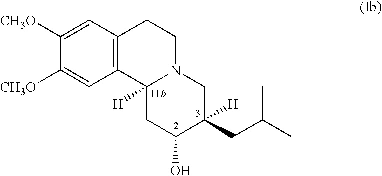
or a pharmaceutically acceptable salt thereof.

6. The method of claim 1, wherein the 3,11b-cis-dihydrotetrabenazine, or a pharmaceutically acceptable salt thereof, is a 2R,3S,11bR isomer of 3,11b-cis-dihydrotetrabenazine having the formula (Ic): 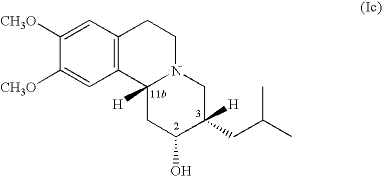
or a pharmaceutically acceptable salt thereof.

7. The method of claim 1, wherein the 3,11b-cis-dihydrotetrabenazine, or a pharmaceutically acceptable salt thereof, is a 2S,3R,11bS isomer of 3,11b-cis-dihydrotetrabenazine having the formula (Id): 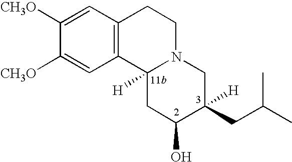
or a pharmaceutically acceptable salt thereof.

8. The method of claim 1, wherein the 3,11b-cis-dihydrotetrabenazine is a free base.

9. The method of claim 1, wherein the pharmaceutically acceptable salt of 3,11b-cis-dihydrotetrabenazine is an acid addition salt.

10. The method of claim 9, wherein the salt is a methane sulphonate salt.
